(19) United States
(12) Patent Application Publication
(10) Pub. No.: US 2022/0262528 A1
(43) Pub. Date: Aug. 18, 2022

(54) METHOD AND APPARATUS WITH ADVERSE DRUG REACTION DETECTION BASED ON MACHINE LEARNING

(71) Applicant: RESEARCH & BUSINESS FOUNDATION SUNGKYUNKWAN UNIVERSITY, Suwon-si (KR)

(72) Inventors: Ji Hwan BAE, Suwon-si (KR); Ju Young SHIN, Suwon-si (KR); Yeon Hee BAEK, Suwon-si (KR)

(73) Assignee: RESEARCH & BUSINESS FOUNDATION SUNGKYUNKWAN UNIVERSITY, Suwon-si (KR)

(21) Appl. No.: 17/673,894

(22) Filed: Feb. 17, 2022

(30) Foreign Application Priority Data
Feb. 17, 2021 (KR) 10-2021-0021407

(51) Int. Cl. G16H 70/40; G06N 20/20; G16H 50/70

(52) U.S. Cl. CPC G16H 70/40; G16H 50/70; G06N 20/20

(57) ABSTRACT

A method that detects adverse drug reactions based on machine learning is provided. The method includes receiving raw data including information on adverse events of a plurality of patients with respect to a target drug; classifying the raw data into first data corresponding to adverse reactions of the target drug, second data corresponding to no adverse reactions of the target drug and drugs similar to the target drug, and third data by implementing a database including information about adverse reactions of the target drug and drugs similar to the target drug based on a predetermined standard; learning a machine learning model by implementing a gold standard dataset including data corresponding to the first data and the second data; and determining a possibility of adverse reactions for the prediction dataset including the data corresponding to the third data by implementing the machine learning model.

### CROSS-REFERENCE TO RELATED APPLICATIONS

This application claims the benefit under 35 USC § 119(a) of Korean Patent Application No. 10-2021-0021407, filed on Feb. 17, 2021, in the Korean Intellectual Property Office, the entire disclosure of which is incorporated herein by reference for all purposes.

### BACKGROUND

### 1. Field

The following description relates to a method and an apparatus with detection of unknown adverse reactions of drugs based on machine learning algorithms.

### 2. Description of Related Art

To collect adverse events caused by drug use worldwide, spontaneous reporting systems were established, and tens of millions of drug-related adverse events have been reported so far. Methods have been developed to detect safety signals pertaining to adverse drug reactions by applying a data mining technique to such a large-scale accumulated database.

However, recent data mining techniques may produce inaccurate safety signals, due to limitations that a dependent calculation method and a threshold are equally applied to specific variables (report case of interest drug-interest adverse event, report case of interest drug-other adverse events, report case of other drugs-interest adverse event, and report case of other drugs-other adverse events) to calculate indicators of safety signals.

In view of these limitations, each regulatory agency may not use only one data mining index, but may use several indices that are complementary to each other. Additionally, due to a decrease in accuracy of the detected safety signals, a utility value of signals in the follow-up work processes, and signal validation and evaluation research, may be reduced, and accordingly, the time consumption may increase.

Attempts to implement artificial intelligence-based algorithms in the healthcare field to handle and analyze big data have continued, and accordingly, there is a demand for a method and an apparatus to detect adverse drug reactions based on machine learning with superior accuracy compared to the typical methods when utilizing spontaneous adverse event report data.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In a general aspect, an adverse drug reaction detection method includes: receiving raw data including information on adverse events of a plurality of patients with regard to a target drug; classifying the received raw data into first data corresponding to adverse drug reactions of the target drug, second data corresponding to no adverse reactions of the target drug and drugs similar to the target drug, and third data; learning a machine learning model by implementing a gold standard dataset including data corresponding to the first data and the second data among the received raw data; and determining a possibility of adverse reactions for a prediction dataset including the data corresponding to the third data among the received raw data by implementing the machine learning model.

The first data, the second data and the third data may be classified based on a database including information about the adverse reactions of the target drug and the drugs similar to the target drug based on a predetermined standard.

The machine learning model may be a learning model that implements one of a gradient boosting machine and a random forest algorithm.

The learning of the machine learning model may include learning the gold standard dataset to be randomly divided into a training dataset and an evaluation dataset according to a predetermined ratio.

The learning of the machine learning model may include first learning the machine learning model by implementing the training dataset; and setting a threshold to have a maximum area under the curve (AUC) of a receiver operating characteristics (ROC) curve with the evaluation dataset for the first learned machine learning model.

The determining of the possibility of adverse reactions for the prediction dataset may include further determining whether there are adverse reactions based on the possibility of adverse reactions of the prediction dataset and the set threshold.

In a general aspect, an adverse drug reaction detection apparatus includes a receiver configured to receive raw data including information on adverse events of a plurality of patients with regard to a target drug; a classifier configured to classify the raw data into first data corresponding to adverse drug reactions of a target drug, second data corresponding to no adverse reactions of a target drug and drugs similar to the target drug, and third data; a learning device configured to learn a machine learning model by implementing a gold standard dataset including data corresponding to the first data and the second data among the received raw data; and a determiner configured to determine a possibility of adverse reactions for a prediction dataset including the data corresponding to the third data among the received raw data by implementing the machine learning model.

The first data, the second data and the third data may be classified based on a database including information about the adverse reactions of the target drug and the drugs similar to the target drug based on a predetermined standard.

The machine learning model may be a learning model that implements one of a gradient boosting machine and a random forest algorithm.

The learning device may be further configured to learn the gold standard dataset to be randomly divided into a training dataset and an evaluation dataset according to a predetermined ratio.

The learning device may be further configured to first learn the machine learning model by implementing the training dataset, and set a threshold to have a maximum area under the curve (AUC) of a receiver operating characteristics (ROC) curve with the evaluation dataset for the first learned machine learning model.

The determiner may be further configured to determine whether there are adverse reactions based on the possibility of adverse reactions of individual data included in the prediction dataset and the set threshold.

Other features and aspects will be apparent from the following detailed description, the drawings, and the claims.

### BRIEF DESCRIPTION OF DRAWINGS

FIG. 1 is a flowchart illustrating a method to detect adverse drug reactions based on machine learning, in accordance with one or more embodiments.

FIG. 2 is a flowchart illustrating an example machine learning method, in accordance with one or more embodiments.

FIG. 3 is a block diagram illustrating an example apparatus to detect adverse drug reactions based on machine learning, in accordance with one or more embodiments.

FIG. 4 is a diagram illustrating an experimental result of an example method to detect adverse drug reactions based on machine learning, in accordance with one or more embodiments.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known after an understanding of the disclosure of this application may be omitted for increased clarity and conciseness, noting that omissions of features and their descriptions are also not intended to be admissions of their general knowledge.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Although terms such as “first,” “second,” and “third” may be used herein to describe various members, components, regions, layers, or sections, these members, components, regions, layers, or sections are not to be limited by these terms. Rather, these terms are only used to distinguish one member, component, region, layer, or section from another member, component, region, layer, or section. Thus, a first member, component, region, layer, or section referred to in examples described herein may also be referred to as a second member, component, region, layer, or section without departing from the teachings of the examples.

Throughout the specification, when an element, such as a layer, region, or substrate is described as being “on,” “connected to,” or “coupled to” another element, it may be directly “on,” “connected to,” or “coupled to” the other element, or there may be one or more other elements intervening therebetween. In contrast, when an element is described as being “directly on,” “directly connected to,” or “directly coupled to” another element, there can be no other elements intervening therebetween.

The terminology used herein is for the purpose of describing particular examples only, and is not to be used to limit the disclosure. As used herein, the singular forms “a,” “an,” and “the” are intended to include the plural forms as well, unless the context clearly indicates otherwise. As used herein, the term “and/or” includes any one and any combination of any two or more of the associated listed items. As used herein, the terms “include,” “comprise,” and “have” specify the presence of stated features, numbers, operations, elements, components, and/or combinations thereof, but do not preclude the presence or addition of one or more other features, numbers, operations, elements, components, and/or combinations thereof.

Unless otherwise defined, all terms, including technical and scientific terms, used herein have the same meaning as commonly understood by one of ordinary skill in the art to which this disclosure pertains and after an understanding of the disclosure of this application. Terms, such as those defined in commonly used dictionaries, are to be interpreted as having a meaning that is consistent with their meaning in the context of the relevant art and the disclosure of this application, and are not to be interpreted in an idealized or overly formal sense unless expressly so defined herein.

Also, in the description of example embodiments, detailed description of structures or functions that are thereby known after an understanding of the disclosure of the present application will be omitted when it is deemed that such description will cause ambiguous interpretation of the example embodiments.

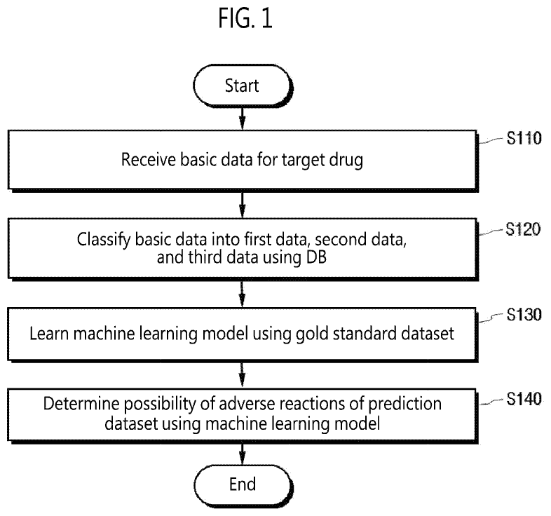

FIG. 1 is a flowchart illustrating an example method of detecting adverse drug reactions based on machine learning algorithms, in accordance with one or more embodiments.

Referring to FIG. 1, in operation S110, an apparatus that detects adverse drug reactions may receive raw data including information pertaining to adverse events of a plurality of patients with respect to a target drug. Herein, it is noted that use of the term ‘may’ with respect to an example or embodiment, e.g., as to what an example or embodiment may include or implement, means that at least one example or embodiment exists where such a feature is included or implemented while all examples and embodiments are not limited thereto.

In an example, the apparatus that detects adverse drug reactions may receive data included in spontaneous adverse event report data through a wired and/or wirelessly connected network, or from a directly connected memory device such as, but not limited to, a universal serial bus (USB), a hard disk drive (HDD), and a solid state drive (SSD).

Additionally, basic or raw data may include information such as, but not limited to, 1) drug information, 2) adverse event information, 3) patient information (for example, gender, age), 4) report information (report type, reporter information, and reporting institution information), etc.

In an example, the apparatus that detects adverse drug reactions may use feature data that is formed by using the number of cases reported as the corresponding adverse events, the number of cases reported as other adverse events, the number of cases reported as the corresponding adverse events in control drugs, the number of cases reported as other adverse events in control drugs, the number of cases by sex, age group, report type, reporter occupation, and reporting institution in a target drug, and codes of a biological institution of the corresponding adverse events based on the adverse event.

Referring again to FIG. 1, in operation S120, the apparatus that detects adverse drug reactions may classify raw data into first data corresponding to adverse drug reactions of a target drug, second data corresponding to no adverse reactions of a target drug and similar drugs, and the remaining third data using a database including information about adverse reactions of the drug thereof and similar drugs which are similar to the drug thereof according to a predetermined standard.

At this time, the database (DB) may be a database including product information related with a drug registered with the Ministry of Food and Drug Safety (MFDS), the US Food and Drug Administration (FDA), and the European Medicines Agency (EMA) and similar drugs classified by main ingredients, target diseases, treatment methods, etc. similar to the drug. In an example, the DB may include information on adverse reactions of the drug and similar drugs.

On the other hand, in the one or more examples, adverse reactions and adverse events may be divided and implemented as follows. The adverse events refer to negative effects of drugs that occur regardless of causality with drugs, and the adverse reactions refer to negative effects that cannot be excluded from causality with drugs.

That is, the apparatus that detects adverse drug reactions may label the raw data or feature data as the first data, the second data, and the third data implementing such a database.

More specifically, the apparatus that detects adverse drug reactions may label data corresponding to the adverse reactions of a target drug stored in the database among the raw data as the first data.

Additionally, the apparatus that detects adverse drug reactions may label data that does not correspond to the adverse reactions of a target drug stored in the database among the raw data and does not correspond to the adverse reactions of similar drugs stored in the database as the second data.

Additionally, the apparatus that detects adverse drug reactions may classify the rest of the raw data other than the first data and the second data as the third data.

Referring again to FIG. 1, in operation S130, the apparatus that detects adverse drug reactions may learn a machine learning model by implementing a gold standard dataset including data corresponding to the first data and the second data among the raw data.

In an example, the gold standard dataset may be a dataset created by collecting data corresponding to the first data and the second data among the raw data.

That is, the apparatus that detects adverse drug reactions may learn a machine learning model by implementing the gold standard dataset to be used to acquire safety signals pertaining to adverse reactions later.

In a non-limiting example, the machine learning model may be a learning model that implements a gradient boosting machine or random forest algorithm.

In an example, the gradient boosting machine algorithm may be a boosting technique that progressively improves a residual error by transmitting an error generated from the result of the previous learning to the next learning, and may form a strong model by binding several decision trees. Additionally, the random forest algorithm may solve an overfitting problem of decision trees, and may be a technique for outputting classification or prediction values using a plurality of decision trees constructed in a training process.

Accordingly, the apparatus that detects adverse drug reactions may implement a decision tree-based machine learning model such as, but not limited to, a gradient boosting machine or random forest algorithm, or the like.

In an example, when the apparatus that detects adverse drug reactions learns the machine learning model, the apparatus may learn the gold standard dataset to be randomly divided into a training dataset and an evaluation dataset according to a predetermined ratio.

In an example, the apparatus that detects adverse drug reactions may learn the gold standard dataset to be randomly classified into a training dataset (75%) and an evaluation dataset (25%).

Additionally, the apparatus that detects adverse drug reactions may adjust the imbalance of the label data by implementing an oversampling technique in order to resolve the imbalance of the label data, which may be one of the factors that hinders the learning of the machine. That is, the apparatus that detects adverse drug reactions may adjust the imbalance of the first dataset and the second dataset included in the training dataset or the evaluation dataset by applying the oversampling technique.

Additionally, the apparatus that detects adverse drug reactions may utilize cross-validation and hyper-parameter tuning techniques to prevent the overfitting of the learning data of the learning model.

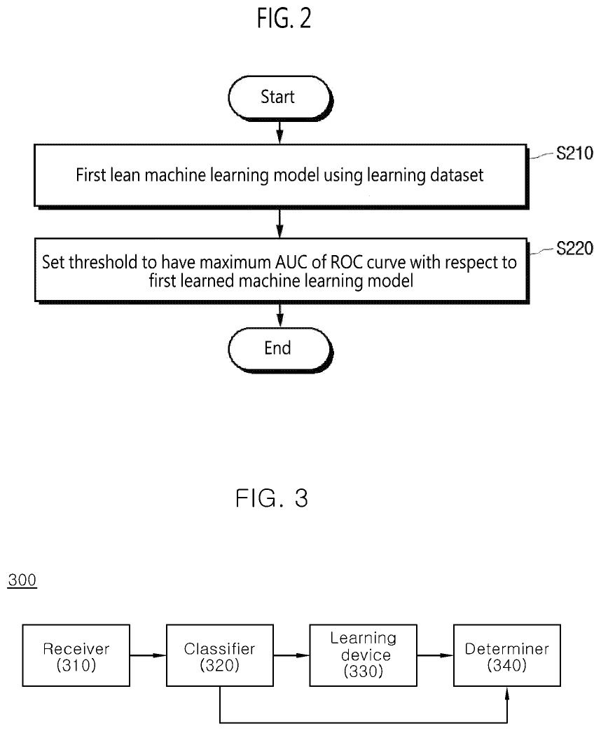

Meanwhile, a method of generating a machine learning model will be described below in detail in the description of FIG. 2.

Referring again to FIG. 1, in operation S140, the apparatus that detects adverse drug reactions may determine the possibility of adverse reactions for the prediction dataset including the data corresponding to the third data among the raw data by using the machine learning model.

In an example, the apparatus that detects adverse drug reactions may implement the prediction dataset as input data, and quantify and determine a possibility of adverse reactions for individual data included in the prediction dataset by implementing a machine learning model.

More specifically, the apparatus that detects adverse drug reactions may calculate and obtain a correlation with a target drug of the adverse events included in the prediction dataset as a probability.

FIG. 4, illustrates an experimental result of a method to detect adverse drug reactions based on machine learning, in accordance with one or more embodiments.

In this experiment, examples that detect adverse events related to nivolumab, an immune anticancer drug, and docetaxel, a cytotoxic anticancer drug, were selected as simulation cases to conduct an experiment.

It can be seen that the performance of the machine learning model (gradient boosting machine and random forest), according to an example, may be overwhelmingly superior to the prediction performance of the previous statistical methods (reporting odds ratio, information component). Additionally, high predictive performance was also shown in predicting safety signals for new drugs such as nivolumab, which do not have a long period of use, which may be considered to indicate high applicability.

Additionally, safety signals that have not been observed in typical statistical methods were observed in the predictive model generated in the examples. Accordingly, the one or more examples may detect important safety signals that cannot be detected by the existing methods.

Since the variables used to generate the input dataset in the one or more examples are variables that may be essentially reported not only in domestic data, but also in data received from overseas, or data received from other countries, the dataset and the method introduced in the one or more examples have a feature that can be utilized even in other overseas data.

FIG. 2 is a flowchart illustrating an example machine learning method, in accordance with one or more embodiments.

Referring to FIG. 2, in operation S210, the example apparatus that detects adverse drug reactions first learns the machine learning model using the training dataset.

That is, the apparatus that detects adverse drug reactions may learn a decision tree-based machine learning model by implementing the generated training dataset.

In operation S220, the example apparatus that detects adverse drug reactions may set a threshold to have a maximum area under the curve (AUC) of a receiver operating characteristics (ROC) curve implementing the evaluation dataset for the first learned machine learning model.

That is, the apparatus that detects adverse drug reactions may generate an optimized predictive model with the best performance by implementing the evaluation dataset.

Accordingly, the apparatus that detects adverse drug reactions may use the area under the curve (AUC) of the receiver operating characteristics (ROC) curve of which an x-axis is 1-specificity and a y-axis is sensitivity as evaluation indexes of the model. That is, the apparatus that detects adverse drug reactions may generate a predictive model by selecting a machine learning algorithm that makes the AUC the largest and an optimized threshold.

In an example, when the apparatus that detects adverse drug reactions determines the possibility of adverse reactions with respect to the prediction dataset, the apparatus may further determine whether there are adverse reactions according to the possibility of adverse reactions of the prediction dataset and a set threshold.

That is, the apparatus that detects adverse drug reactions may quantify a possibility of adverse reactions for individual data included in the prediction dataset by implementing the machine learning model, and determine whether there are adverse reactions that have not been previously known by comparing the quantified possibility of adverse reactions with a predetermined threshold. For example, the apparatus that detects adverse drug reactions may determine, as the adverse reactions, when the possibility of adverse reactions is greater than the threshold.

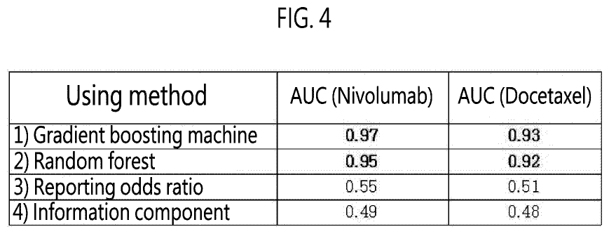

FIG. 3 is a block diagram illustrating an apparatus that detects adverse drug reactions based on machine learning, in accordance with one or more embodiments.

Referring to FIG. 3, an apparatus 300 that detects adverse drug reactions based on machine learning, in accordance with one or more embodiments, may include a receiver 310, a classifier 320, a learning device 330, and a determiner 340.

The apparatus 300 that detects adverse drug reactions based on machine learning, in accordance with one or more embodiments, may be mounted on various types of computing devices such as, but not limited to, smartphones, tablets, desktop personal computers (PCs), notebook PCs, and server computers.

The receiver 310 may receive raw data including information on adverse reactions of a plurality of patients with respect to a target drug.

The classifier 320 may classify the raw data into first data corresponding to adverse drug reactions of a target drug, second data corresponding to no adverse reactions of a target drug and drugs that are similar to the target drug, and the remaining third data using a database including information about adverse reactions of the target drug thereof and drugs which are similar to the target drug thereof according to a predetermined standard.

The learning device 330 may learn a machine learning model by implementing a gold standard dataset including data corresponding to the first data and the second data among the raw data.

In an example, the machine learning model may be a learning model that implements a gradient boosting machine or random forest algorithm.

In an example, the learning device 330 may learn the gold standard dataset to be randomly divided into a training dataset and an evaluation dataset according to a predetermined ratio.

In an example, the learning device 330 may first learn the machine learning model by implementing the training dataset and set a threshold to have a maximum area under the curve (AUC) of a receiver operating characteristics (ROC) curve using the evaluation dataset for the first learned machine learning model.

The determiner 340 may determine a possibility of adverse reactions for the prediction dataset including the data corresponding to the third data among the raw data by implementing the machine learning model.

In an example, the determiner 340 may further determine whether there are adverse reactions according to the possibility of adverse reactions of individual data included in the prediction dataset and the set threshold.

The receiver 310, the classifier 320, the learning device 330, the determiner 340, as well as the remaining apparatuses, units, modules, devices, and other components, described herein may be implemented by hardware components and software components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions in the specification, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media. Examples of a non-transitory computer-readable storage medium include read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD−Rs, CD+Rs, CD−RWs, CD+RWs, DVD-ROMs, DVD−Rs, DVD+Rs, DVD−RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An adverse drug reaction detection method, the method comprising:
receiving raw data including information on adverse events of a plurality of patients with regard to a target drug;
classifying the received raw data into first data corresponding to adverse drug reactions of the target drug, second data corresponding to no adverse reactions of the target drug and drugs similar to the target drug, and third data;
learning a machine learning model by implementing a gold standard dataset including data corresponding to the first data and the second data among the received raw data; and
determining a possibility of adverse reactions for a prediction dataset including the data corresponding to the third data among the received raw data by implementing the machine learning model.

2. The method of claim 1, wherein the first data, the second data and the third data are classified based on a database including information about the adverse reactions of the target drug and the drugs similar to the target drug based on a predetermined standard.

3. The method of claim 1, wherein the machine learning model is a learning model that implements one of a gradient boosting machine and a random forest algorithm.

4. The method of claim 1, wherein the learning of the machine learning model comprises learning the gold standard dataset to be randomly divided into a training dataset and an evaluation dataset according to a predetermined ratio.

5. The method of claim 4, wherein the learning of the machine learning model comprises:
first learning the machine learning model by implementing the training dataset; and
setting a threshold to have a maximum area under the curve (AUC) of a receiver operating characteristics (ROC) curve with the evaluation dataset for the first learned machine learning model.

6. The method of claim 5, wherein the determining of the possibility of adverse reactions for the prediction dataset comprises further determining whether there are adverse reactions based on the possibility of adverse reactions of the prediction dataset and the set threshold.

7. An adverse drug reaction detection apparatus, the apparatus comprising:
a receiver configured to receive raw data including information on adverse events of a plurality of patients with regard to a target drug;
a classifier configured to classify the raw data into first data corresponding to adverse drug reactions of a target drug, second data corresponding to no adverse reactions of a target drug and drugs similar to the target drug, and third data;
a learning device configured to learn a machine learning model by implementing a gold standard dataset including data corresponding to the first data and the second data among the received raw data; and
a determiner configured to determine a possibility of adverse reactions for a prediction dataset including the data corresponding to the third data among the received raw data by implementing the machine learning model.

8. The apparatus of claim 7, wherein the first data, the second data and the third data are classified based on a database including information about the adverse reactions of the target drug and the drugs similar to the target drug based on a predetermined standard.

9. The apparatus of claim 7, wherein the machine learning model is a learning model that implements one of a gradient boosting machine and a random forest algorithm.

10. The apparatus of claim 7, wherein the learning device is further configured to learn the gold standard dataset to be randomly divided into a training dataset and an evaluation dataset according to a predetermined ratio.

11. The apparatus of claim 10, wherein the learning device is further configured to first learn the machine learning model by implementing the training dataset, and set a threshold to have a maximum area under the curve (AUC) of a receiver operating characteristics (ROC) curve with the evaluation dataset for the first learned machine learning model.

12. The apparatus of claim 11, wherein the determiner is further configured to determine whether there are adverse reactions based on the possibility of adverse reactions of individual data comprised in the prediction dataset and the set threshold.
